(19) United States
(12) Patent Application Publication
(10) Pub. No.: US 2006/0167006 A1
(43) Pub. Date: Jul. 27, 2006

(54) Method of treatment

(76) Inventors: Helmut Friess, Eppelheim (DE); Joerg Kleeff, Heidelberg (DE)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/386,141

(22) Filed: Mar. 22, 2006

Related U.S. Application Data
(63) Continuation of application No. 10/955,838, filed on Sep. 30, 2004, now abandoned.
(60) Provisional application No. 60/508,357, filed on Oct. 3, 2003.
(60) Provisional application No. 60/588,012, filed on Jul. 14, 2004.

(51) Int. Cl. A61K 31/506

(52) U.S. Cl. 514/252.18

(57) ABSTRACT

A method of treating a warm-blooded animal suffering from pancreatic cancer which. comprises administering to said animal in need of such a treatment a dose, effective against said disease, of 4-(4-methylpiperazin-1-ylmethyl)-N-[4-methyl-3-(4-pyridin-3-yl)pyrimidin-2-yl-amino)phenyl]-benzamide or a pharmaceutically acceptable salt thereof.

This application claims benefit of U.S. provisional application No. 60/508,357, filed Oct. 3, 2003 and U.S. provisional application No. 60/588,012, filed Jul. 14, 2004, the contents of which are incorporated herein by reference.

The invention relates to the use of 4-(4-methylpiperazin-1-ylmethyl)-N-[4-methyl-3-(4-pyridin-3-yl)pyrimidin-2-ylamino)phenyl]-benzamide. (hereinafter referred to as “Compound I”) or a pharmaceutically acceptable salt thereof for the manufacture of pharmaceutical compositions for the treatment of pancreatic cancer, especially pancreatic ductal adenocarcinoma, to the use of Compound I or a pharmaceutically acceptable salt thereof in the treatment of pancreatic cancer, to a method of treating warm-blooded animals including mammals, especially humans, suffering from pancreatic cancer by administering to said animal in need of such a treatment a dose effective against said disease of Compound I or a pharmaceutically acceptable salt thereof.

Pancreatic cancer has an incidence of about 10 cases/100,000 persons, it is the fourth to fifth leading cause of cancer-related deaths in the Western world. Most of the newly diagnosed patients present at an already unresectable tumor stage. The 5-year survival rate of these patients is less than 1% and the median survival time is approximately 5-6 months after tumor detection. One of the reasons for this is that conventional oncological strategies, such as chemotherapy, radiotherapy, anti-hormonal modalities or systemic use of monoclonal antibodies, have not achieved significant improvement in the survival of pancreatic cancer patients. Pancreatic ductal adenocarcinoma is the most common form of pancreatic cancer accounting for 75 to 95 % of all pancreatic cancers.

Human pancreatic cancers over-express a number of important tyrosine kinase growth factor receptors and their ligands, such as those belonging to the epidermal growth factor (EGF), fibroblast growth factor (FGF), insulin-like growth factor (IGF-1), and vascular endothelial growth factor (VEGF) families. In addition, expression of both PDGF and PDGF receptors (PDGFRs) has been observed in pancreatic cancer. It is thought that these growth factors act in an autocrine and/or paracrine manner to stimulate pancreatic cancer growth. Binding of growth factors to their receptors results in receptor autophosphorylation and subsequent signal transduction via an array of different molecules.

Compound I is 4-(4-methylpiperazin-1-ylmethyl)-N-[4-methyl-3-(4-pyridin-3-yl)pyrimidin-2-ylamino)phenyl]-benzamide having the following formula

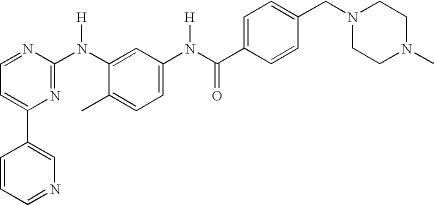

Compound I free base, its acceptable salts thereof and its preparation are disclosed in the European granted patent 0564409 hereby incorporated by reference. Compound I free base corresponds to the active moiety.

The monomethanesulfonic acid addition salt of Compound I (hereinafter referred to as “Salt I”) and a preferred crystal form thereof (the beta crystal form) are described in PCT patent application WO99/03854 hereby incorporated by reference.

Surprisingly, it was found that Compound I is particularly useful for the treatment of pancreatic cancer.

By “pancreatic cancer” is meant a disease in which cancer cells are found in the tissues of the pancreas, e.g. pancreatic ductal adenocarcinoma.

The invention relates in the use of Compound I or a pharmaceutically acceptable salt thereof, e.g. Salt I, as a drug against pancreatic cancer.

The present invention pertains to the use of Compound I or a pharmaceutically acceptable salt thereof for the manufacture of a medicament for the treatment of pancreatic cancer.

The pharmaceutical compositions according to the present invention can be prepared in a manner known per se and are those suitable for enteral, such as oral or rectal, and parenteral administration to warm-blooded animals, e.g. human, comprising a therapeutically effective amount of at least one pharmacologically active ingredient, alone or in combination with one or more pharmaceutically acceptable carriers, especially suitable for enteral or parenteral application. The preferred route of administration of the dosage forms of the present invention is orally.

The invention also relates to a method of treating a warm-blooded animal, e.g. a human, having pancreatic cancer comprising administering to said animal in need for such a treatment Compound I in a quantity which is therapeutically effective against pancreatic cancer.

The invention relates to a method for administering to a human subject suffering from pancreatic cancer an acid addition salt of 4-(4-methylpiperazin-1-ylmethyl)-N-[4-methyl-3-(4-pyridin-3-yl)pyrimidin-2-ylamino)phenyl]-benzamide, e.g. Salt I.

According to the present invention, the pancreatic cancer is preferably the pancreatic ductal adenocarcinoma.

The person skilled in the pertinent art is fully enabled to select relevant test models to prove the beneficial effects mentioned herein on pancreatic cancer. The pharmacological activity of such a compound may, for example, be demonstrated by means of the Examples described below, by in vitro tests and in vivo tests or in suitable clinical studies. Suitable clinical studies are, for example, open label non-randomized, dose escalation studies in patients with pancreatic cancer. The efficacy of the treatment is determined in these studies, e.g. by evaluation of the tumor sizes every 4 weeks.

The effective dosage of Compound I may vary depending on the pharmaceutical composition employed, on the mode of administration, the type of the pancreatic cancer being treated or its severity. The dosage regimen is selected in accordance with a variety of further factors including the renal and hepatic function of the patient. A physician, clinician or veterinarian of ordinary skill can readily determine and prescribe the effective amount of compounds required to prevent, counter or arrest the progress of the condition.

Depending on age, individual condition, mode of administration, and the clinical picture in question, effective doses, for example daily doses of Compound I or a pharmaceutically acceptable salt thereof, e.g. Salt I, corresponding to 100 to 1000 mg of the free base as active moiety, e.g. 200 to 800 mg, 200 to 600 mg, e.g. 400 mg, are administered to warm-blooded animals of about 70 kg body weight. Preferably, the warm-blooded animal is a human. For patients with an inadequate response to daily doses, dose escalation can be safely considered and patients may be treated as long as they benefit from treatment and in the absence of limiting toxicities.

The invention relates also to a method for administering to a human subject suffering from pancreatic cancer, Compound I or a pharmaceutically acceptable salt thereof, e.g. Salt I, which comprises administering a pharmaceutically effective amount of Compound I or a pharmaceutically acceptable salt thereof, e.g. Salt I, to said human subject once daily for a period exceeding 3 months. The invention relates especially to such method wherein a daily dose of 400 to 800 mg preferably 800 mg, of Compound I is administered to an adult.

The present invention does not cover the use of 4-(4-methylpiperazin-1-ylmethyl)-N-[4-methyl-3-(4-pyridin-3-yl)pyrimidin-2-ylamino)phenyl]-benzamide or a pharmaceutically acceptable salt thereof for enhancing the effect of radioimmunotherapy of pancreatic cancer, or for treating pancreatic cancer in patients subject to radioimmunotherapy, or when used in combination with a radioimmunoconjugate for treatment of pancreatic cancer. Specifically, the present invention disclaims the use of 4-(4-methylpiperazin-1-ylmethyl)-N-[4-methyl-3-(4-pyridin-3-yl)pyrimidin-2-ylamino)phenyl]-benzamide or a pharmaceutically acceptable :salt thereof, for the treatment of pancreatic cancer when radioimmunotherapy is necessary and the compound is administered during, before and/or after the radioimmunotherapy treatment period as disclosed in application 60/397,347 filed Jul. 19, 2002 or PCT IB03/03257 filed Jul. 17, 2003.

### EXAMPLE 1

### Effect of Compound I on the Growth of TAKA-1 Cells and Pancreatic Cancer Cell Lines:

The publication entitled “The stem cell factor-c-kit system and mast cells in human pancreatic cancer” by Esposito et al, Laboratory Investigation 2002, Vol. 82, No. 11, p 1481-92 is hereby incorporated by reference.

Cell culture and proliferation assay: The following materials are purchased: FCS, RPMI and Dulbecco's modified Eagle medium (DMEM), trypsin-EDTA solution, penicillin-streptomycin solution from Biochrom K G (Berlin, Germany). All the other reagents are from Sigma Chemical Co. (St. Louis, Mo., USA). ASPC-1, MIA PaCA-2, human pancreatic cell lines are obtained from American Type Culture Collection (Rockville, Md.). Compound I is kindly provided by Novartis Pharma AG (Basel, Switzerland). T3M4 human pancreatic cell lines are a gift from Dr. R. S. Metzgar (Durham, N.C., USA). TAKA-1 immortalized Syrian golden hamster pancreatic ductal cells are a gift from Prof. P. Pour (University of Nebraska, Omaha, Nebr., USA). Human pancreatic cancer cell lines are routinely grown in DMEM (MIA-PaCA-2) or RPMI (ASPC-1, T3M4) supplemented with 10% FCS, 100 U/ml penicillin and 100 μg/ml streptomycin (complete medium).

TAKA-1 cells are grown in RPMI supplemented with 10% FCS, 100 U/ml penicillin and 100 μg/ml streptomycin. Cells are seeded overnight at a density of 5000 cells/well in 96-well plates and the proliferation assay is performed as described above. To assess the effect of Compound I on cell growth, TAKA-1 cells and pancreatic cancer cell lines are incubated in the absence (control) or presence of the indicated concentrations of Compound I. Cell growth is determined after 72 hours by the MTT colorimetric assay. After 72 hours, 3-(4,5-methylthiazol-2-yl)-2,5-diphenyl-tetrazolium bromide (MTT) is added (50 μg/well) for 4 hours. Cellular MTT is solubilized with acidic isopropanol and the optical density is measured at 570 nm using a plate reader (Dynatech MR 7000). All experiments are performed in triplicates.

Statistical analysis: Results are expressed as mean±SD. For statistical analysis, the Student t-test is used. Significance is defined as p<0.05.

Compound I inhibits the growth of TAKA-1 cells and of the three pancreatic cancer cell lines tested in this study in a dose-dependent fashion. Minimal threshold effects of −4% to −9% (p>0.05) occurred at a concentration of 5 μM Compound I. Maximal inhibitory effects of −77% to −90% are observed in these cells at a concentration of 75 pM Compound I (p<0.001) (Table 1). In this study, the administration of Compound I to three pancreatic cancer cell lines resulted in a significant growth inhibition. The exact mechanisms of this effect are not known.

TABLE 1
Effect of Compound I on the growth of TAKA-1 cells and pancreatic
cancer cell lines. TAKA-1, ASPC-1, MIA PaCA-2 and T3M4 cells
are incubated in the absence (control) or presence of Compound I
(in μM) for 72 h. Cell growth is measured by the MTT assay.
Percent growth inhibition is determined by comparison
with control cell growth.

| Compound I in μM | 0.5 | 5 | 25 | 75 |
|---|---|---|---|---|
| TAKA-1 | −4.2 | 0.2 | −70.9 | −90.8 |
| | −4.6 | 1.4 | −65 | −91.5 |
| | 26 | −13.7 | −67.9 | −90.6 |
| Aspc-1 | 12.9 | −12.9 | −61.1 | −85.6 |
| | 9.1 | −4.8 | −53.9 | −80.2 |
| | −3.3 | −3.7 | −35.1 | −66.3 |
| Mia-Paca-2 | −6.7 | −11.8 | −65.8 | −86.6 |
| | 21.8 | −20.8 | −65 | −85 |
| | 2.1 | 4 | −39.4 | −76.2 |
| T3M4 | 12.9 | −12.9 | −61.1 | −85.6 |
| | 9.1 | −4.8 | −53.9 | −80.2 |
| | −3.3 | −3.8 | −35.1 | −66.3 |

### EXAMPLE 2

### Effect of Compound I on Pancreatic Cancer Cell Growth

Material and Methods: RPMI-1640 DMEM, trypsin-EDTA, and penicillin-streptomycin are purchased from Invitrogen (Karlsruhe, Germany); FBS from PAN Biotech (Aidenbach, Germany); human recombinant PDGF, IGF-1 and FGF-2 from R&D Systems (Abingdon, United Kingdom), and EGF from Upstate Biotechnology (Hamburg, Germany). Phospho-PDGFR-beta (tyr857) and P-EGFR (tyr1173) polyclonal antibodies are purchased from Santa Cruz Biotechnology, Inc. (Santa Cruz, Calif., USA). Phospho-p44/42 MAPK (Thr202/Tyr204) antibodies are purchased from Cell Signaling Technology (Frankfurt, Germany), anti-rabbit IgG HRPO-linked antibodies and ECL immunoblotting detection reagents from Amersham Biosciences (Amersham Life Science, Amersham, UK), and anti-goat IgG-HPRO peroxidase linked antibodies from Santa Cruz Biotechnology, Inc. (Santa Cruz, Calif., USA). Complete mini-EDTA-free protease inhibitor cocktail tablets and Annexin-V-Fluos are purchased from Roche GmbH (Mannheim, Germany). All other reagents are from Sigma Chemical Company (Taufkirchen, Germany).

Cell culture and MTT assay: Human pancreatic cancer cell lines are routinely grown in DMEM (Colo-357 and Mia-PaCa-2) or RPMI (Aspc-1, BxPc-3, Capan-1, and T3M4) supplemented with 10% FBS, 100 U/ml penicillin, and 100 μg/ml streptomycin (complete medium). To assess cell proliferation, the MTT test is employed. Briefly, cells are seeded at a density of 5000 cells/well in 96-well plates, grown overnight and exposed to Compound I alone or in combination with growth factors. After 48 or 72 hours of incubation, 3-(4,5-methylthiazol-2-yl)-2,5-diphenyl-tetrazolium bromide (MTT) is added (50 μg/well) for 4 hours. Formazan products are solubilized with acidic isopropanol, and the optical density is measured at 570 nm. To determine the GI₅₀ of Compound I (the concentration that causes 50% growth inhibition), graded concentrations of Compound I are added to triplicate wells and GI₅₀ is calculated using 100×(T−T₀)/(C−T₀)=50. T is the. optical density of the test well, after a 48-hour period of exposure to Compound I, e.g. Salt I, T₀ is the optical density at time zero, and C is the control optical density after 48 hours. All experiments are performed in triplicate.

FACS analysis of cell death and cell cycle: 10⁵ pancreatic tumor cells are seeded into 6-well plates in 1% FBS-containing medium, allowed to adhere overnight and then treated with corresponding GI₅₀ concentrations of Compound I. To analyze cell cycle distribution, cells are collected after 48 hours of incubation, washed with PBS and resuspended in 0.5 ml of hypotonic PI buffer (5 μg/ml propidium iodide, 0.1% Triton X100 and 0.1% sodium citrate), stored overnight at 4° C. and then analyzed by flow cytometry using BD-LSR (Becton Dickinson and Company, New York, USA). The resulting DNA histograms are interpreted using the Cell Quest Pro software (Becton Dickinson and Company, New York, USA). To determine the degree of cell death, cells are collected after 12, 24 and 48 hours of exposure to G1₅₀ of Compound I and are washed and stained with Annexin-V-FITC (apoptotic death) or PI (necrotic death) according to the manufacturer's instructions (Roche, Mannheim, Germany).

Western blot analysis: Cell culture monolayers are washed twice with ice-cold PBS and lysed with buffer containing Tris-HCl (50 mM, pH 7.4), NP-40 (1%), Na-deoxycholate (0.25%), NaCl (150 mM), EDTA (1 mM), PMSF (1 mM), Na₃VO₄ (1 mM), NaF (1 mM) and one tablet of complete mini-EDTA-free protease inhibitor cocktail (in 10 ml buffer). Protein concentration is determined by the BCA protein assay (Pierce Chemical Co., Rockford, Ill., USA). 30 μg of cell lysates are separated on SDS-polyacrylamide gels and electroblotted onto nitrocellulose membranes. Membranes are then incubated in blocking solution (5% nonfat-milk in 20 mM Tris-HCl, 150 mM NaCl, 0.1% Tween-20) (TBS-T), followed by incubation with the indicated antibodies at 4° C. overnight. The membranes are then washed in TBS-T and incubated with HRPO-conjugated secondary antibodies for 1 hour at room temperature. Antibody detection is performed with an enhanced chemiluminescence reaction. Statistical analysis: Results are expressed as mean±SEM. For statistical analysis, the Student's t-test is used. Significance is defined as p<0.05.

Results

Determination of the GI₅₀ Concentration of Compound I in Pancreatic Cancer Cells

To determine the GI₅₀ concentration of Compound I pancreatic cancer cells grown in 10% FBS-containing medium are exposed to different doses of Compound I. As seen from Table I, Compound I inhibits the growth of all tested pancreatic cancer cells in a dose-dependent manner. The concentrations of Compound I required to inhibit cell growth by 50% (GI₅₀) are in the range of 17-31.5 μM, with Colo-357 cells being the most sensitive (17 μM), and Aspc-1 cells the most resistant (31.5. μM). The GI₅₀ for pancreatic cells appeared to be higher than the GI₅₀ for other reported cancer cells. Since effects of Compound I are reported to depend on serum concentration, the GI₅₀ of Compound I under low serum conditions (1% FCS). is tested. GI₅₀ concentrations range between 9 and 20 μM, with Mia-PaCa-2 cells being the most sensitive (9 μM), and Aspc-1 cells the most resistant (20 μM) (Table I). Increasing concentration of growth factors in serum increases the resistance of pancreatic cancer cells to Compound I.

TABLE I
GI₅₀ concentration of Compound I in different cancer cell
lines in comparison to Compound I plasma concentrations.

| cancer cell lines | Compound I (μM) |
|---|---|
| Aspc-1^{#} | 31.5 |
| BxPc-3^{#} | 21 |
| Capan-1^{#} | 19 |
| Colo-357^{#} | 17 |
| Mia-PaCa-2^{#} | 26 |
| T3M4^{#} | 25 |
| Lung cancer (6 cell lines) | ˜5 |
| Lung cancer (A549 cell line) | 2-3 |
| colorectal cancer (HT29) | 6 |
| CML (K562 cell lines) | 0.56 |
| Compound I plasma levels | 0.17-5.68 |

^{#}as determined in complete medium.

Mechanism of Compound I Action on Pancreatic Cancer Cells

The contribution of cytotoxic and cytostatic components to compound I-induced growth inhibition. First, propidium iodide staining of cells is performed in order to determine whether cell death occurred in the treated cultures. Compound I has potent toxic effects towards pancreatic cancer cells: 33.7±16.5% in Mia-PaCa-2 and 26.8% +16.5% in T3M4 cultures as compared to 10.7% ±2.5% and 5.7% ±2.8% in control cultures, respectively (p<0.05). Simultaneous annexin V/propidiuin iodide staining is employed to clarify the type of cell death taking place in the cultures. Progressive accumulation. of annexin V-positive cells is observed, the role of apoptosis as a possible mechanism of Compound I-induced cell death is not further supported by the results of PI staining of nuclear DNA (not shown). Cell cycle analysis of Compound I-treated pancreatic cancer cell lines is performed, which does not reveal any significant changes in the cell cycle pattern after 48 hours of incubation when compared to untreated cells. Apparently, Compound I treatment causes membrane alterations, leading to the layer flipping and phosphatidylserine translocation, but does not induce caspase-dependent DNA fragmentation. The exact mechanisms of the Compound I-induced toxicity towards pancreatic tumor cells remain to be determined.

Effects of Compound I on Growth Factor-Induced Proliferation of Pancreatic Cancer Cells

Mia-PaCa-2 and T3M4 cell lines are exposed to different growth factors (PDGF, EGF, FGF-2, IGF-1) in the absence or presence of Compound I (at GI₅₀ concentration) for 72 hours and cell growth is assessed by MTT assays (not shown). EGF-induced proliferation of both Mia-PaCa-2 and T3M4 cell lines in a dose-dependent manner is seen with maximal effects of +72% ±7.5% (Mia-PaCa-2) and +52% ±23% (T3M4). Interestingly, Compound I partially blocks EGF-induced cell growth, with maximal effects reduced to +29% ±15% (Mia-PaCa-2) and +16% ±24% (T3M4) following Compound I addition (not shown). FGF-2 markedly stimulates cell growth in Mia-PaCa-2 cell lines with maximal effects of +79% ±6%, and only slightly stimulated T3M4 cell lines, with maximal effects of +7% ±4%. Compound I also partly blocked FGF-2 induced cell growth, with maximal effects reduced to +28% ±7% (Mia-PaCa-2) and 2% ±3% (T3M4). Both cell lines are less sensitive to IGF-1 stimulation compared with EGF and FGF-2, with maximal effects of +24% ±2% in Mia-PaCa-2 and +17% ±4% in. T3M4. Compound I has no significant inhibitory effects on IGF-1-induced proliferation. PDGF does not induce cell proliferation in Mia-PaCa-2 or T3M4 cell lines. Furthermore, in three additional cell lines (BxPc-3, Colo-357 and Capan-1), PDGF also has no effect on cell growth. Although PDGF-mediated growth pathways do not seem to play a role in pancreatic cancer, the above data demonstrate the ability of Compound I to interfere with other growth-stimulatory signaling pathways, such as EGF and FGF-2.

Effects of Compound I on Growth Factor-Induced Receptor and MAP Kinase Activation.

A common cellular response to a variety of extracellular signals involves the phosphorylation of corresponding receptors and activation of the MAPK pathway. The partial obstruction of EGF, FGF-2, and IGF-1 signaling by Compound I is due to inhibition of receptor- and/or MAPK phosphorylation. Treatment of growth factor-conditioned Mia-PaCa-2 and T3M4 cells with Compound I does not inhibit activation of either MAPK or the EGFR. PDGF does not induce PDGF and MAPK kinase phosphorylation in those cell lines (data not shown).

Pancreatic cancer cells, resistant to growth-induction through Compound I-sensitive c-kit and PDGF pathways, are still responsive to the inhibitory effects of Compound I, although at high GI₅₀ concentrations.

Protein kinases play a crucial role in signal transduction as well as in cellular proliferation, differentiation, and various regulatory mechanisms. Deregulation of those signaling pathways is frequent during malignant transformation. Compound I target c-kit and its ligand stem cell factor (SCF) have been shown to be expressed in pancreatic cancer cells. It has been shown that SCF has no significant effects on pancreatic cancer cell growth, whereas Compound I inhibits pancreatic cancer cell growth (Example 1). Compound I may exert its effect on pancreatic cancer cell growth through c-kit-independent pathways.

In pancreatic cancer, a variety of other growth factors that signal through tyrosine kinase receptors are also expressed at increased levels. For example, the presence of EGFs, FGFs, PDGFs, and IGFs and their respective receptors has been observed in pancreatic cancer, and these growth factors are thought to contribute to its malignant phenotype. For example, it has been reported that the mRNA levels of EGF and EGFR are markedly increased in pancreatic cancer tissues in comparison with the normal pancreas, suggesting that the co-expression of EGFR and its ligands may contribute to the aggressiveness of human pancreatic cancer. FGF and its receptors are also over-expressed in pancreatic cancer tissues and cell lines, and cell growth, cell adhesion and invasion are modulated by fibroblast growth factors in pancreatic cancer cell lines. IGF-1 and its receptor IGF1R are also over-expressed in pancreatic cancer, and they also have the potential to stimulate pancreatic cancer cell growth.

Compound I has dose-dependent inhibitory effects in all six tested pancreatic cancer cell lines, with GI₅₀ in the range of 17-31.5 μM (10% FCS) and 9-20 μM (1% FCS). Notably, these concentrations are -relatively high compared with the concentrations sufficient for the inhibition of AbI and c-kit in other tumors.

In cell cycle analysis, no cell cycle change and no apoptosis is caused by Compound I. It has been reported that Compound I did not induce apoptosis in serum-containing medium, and it has been suggested that this is because the cells may be more resistant under these conditions, and serum can persistently activate MAP kinase. Based on these data, cells are treated with Compound I in both complete medium (10% FCS) and 1% FCS medium. No apoptotic cell death is observed under either of these serum concentrations, yet prolongation of Compound I treatment lead to more cell death under these conditions. In addition, more Annexin-V-positive cells are also observed, suggesting that Compound I can damage the integrity of the cell membrane, causing phosphatidylserine translocation to the outer surface of the cells, and at a late stage, causing cell death without significant apoptosis. Compound I inhibits pancreatic cells growth, but these effects are not mediated through blockage of the PDGF receptor tyrosine kinase, since PDGF did not stimulate pancreatic cancer cell growth and did not lead to MAP kinase or PDGF receptor phosphorylation. Compound I partially but not specifically blocks EGF, IGF-1 and FGF-2 mitogenic pathways, which have the potential to stimulate pancreatic cancer cell growth.

### EXAMPLE 3

### Capsules with 4-[(4-methyl-1-piperazin-1-ylmethyl)-N-[4-methyl-3-[[4-(3-pyridinyl)-2-pyrimidinyl]amino]phenyl]benzamide monomethanesulfonate, beta crystal form

Capsules containing 119.5 mg of Salt I corresponding to 100 mg of Compound I (free base) as active moiety are prepared in the following composition:

| | | | |
|---|---|---|---|
| Composition: | Salt I | 119.5 | mg |
| | Cellulose MK GR | 92 | mg |
| | Crospovidone XL | 15 | mg |
| | Aerosil 200 | 2 | mg |
| | Magnesium stearate | 1.5 | mg |
| | | 230 | mg |

The capsules are prepared by mixing the components and filling the mixture into hard gelatin capsules, size 1.

### EXAMPLE 3

### Capsules with 4-[(4-methyl-1-piperazin-1-ylmethyl)-N-[4-methyl-3-[[4-(3-pyridinyl)-2-pyrimidinyl]amino]phenyl]benzamide monomethanesulfonate, beta crystal form

Capsules containing 119.5 mg of Salt I corresponding to 100 mg of Compound I (free base) as active moiety are prepared in the following composition:

| | | | |
|---|---|---|---|
| Composition: | Salt I | 119.5 | mg |
| | Avicel | 200 | mg |
| | PVPPXL | 15 | mg |
| | Aerosil | 2 | mg |
| | Magnesium stearate | 1.5 | mg |
| | | 338.0 | mg |

The capsules are prepared by mixing the components and filling the mixture into hard gelatin capsules, size 1.

1. A method of treating a warm-blooded animal suffering from pancreatic cancer which comprises administering to said animal in need of such a treatment a dose, effective against said disease, of 4-(4-methylpiperazin-1-ylmethyl)-N-[4-methyl-3-(4-pyridin-3-yl)pyrimidin-2-ylamino)phenyl]-benzamide or a pharmaceutically acceptable salt thereof.

2. The method according to claim 1 wherein the pancreatic cancer is a pancreatic ductal adenocarcinoma.

3. The method according to claim 1 wherein a daily dose of 4-(4-methylpiperazin-1-ylmethyl)-N-[4-methyl-3-(4-pyridin-3-yl)pyrimidin-2-ylamino)phenyl]-benzamide or a pharmaceutically acceptable salt thereof corresponding to 100-1000 mg of 4-(4-methylpiperazin-1-ylmethyl)-N-[4-methyl-3-(4-pyridin-3-yl)pyrimidin-2-ylamino)phenyl]-benzamide free base is administered to a warm blooded animal.

4. The method according to claim 3 wherein 4-(4-methylpiperazin-1-ylmethyl)-N-[4-methyl-3-(4-pyridin-3-yl)pyrimidin-2-ylamino)phenyl]-benzamide or a pharmaceutically acceptable salt thereof is administered for a period exceeding 3 months.

5. The method according claim 1 wherein the 4-(4-methylpiperazin-1-ylmethyl)-N-[4-methyl-3-(4-pyridin-3-yl)pyrimidin-2-ylamino)phenyl]-benzamide is in the form of the monomethanesulfonate salt.

6. The method according to claim 5 wherein the monomethane sulfonate salt of 4-(4-methylpiperazin-1-ylmethyl)-N-[4-methyl-3-(4-pyridin-3-yl)pyrimidin-2-ylamino)phenyl]-benzamide is in the beta crystal form.
